(19) United States
(12) Patent Application Publication
(10) Pub. No.: US 2010/0129810 A1
(43) Pub. Date: May 27, 2010

(54) METHODS AND SYSTEMS FOR NUCLEIC ACID SEQUENCING VALIDATION, CALIBRATION AND NORMALIZATION

(75) Inventors: Douglas P. GREINER, Fremont, CA (US); Carmen GJERSTAD, Millbrae, CA (US); Janet S. ZIEGLE, Berkeley, CA (US); Lee W. JONES, Hayward, CA (US); Min-Yi SHEN, Mountain View, CA (US); Chen-Shan CHIN, San Leandro, CA (US); Heinz BREU, Palo Alto, CA (US)

Correspondence Address: O'BRIEN JONES, PLLC, 1951 Kidwell Drive, Suite 550 B, Tyson's Corner, VA, 22182, US

(73) Assignee: LIFE TECHNOLOGIES CORPORATION, Carlsbad, CA (US)

(21) Appl. No.: 12/555,549

(22) Filed: Sep. 8, 2009

Related U.S. Application Data
(60) Provisional application No. 61/094,785, filed on Sep. 5, 2008.

(51) Int. Cl. C12Q 1/68

(52) U.S. Cl. 435/6

(57) ABSTRACT

A system for performing quality control for nucleic acid sample sequencing is disclosed. The system has a set of solid supports, each support having attached thereto a plurality of nucleic acid sequences. The set has plural groups of solid supports and each group contains solid supports having the same nucleic acid sequences attached thereto. The nucleic acid sequences of each group differ from each other. The nucleic acid sequences are synthetically derived. A method of preparing a quality control for performing nucleic acid sample sequencing and a method of validating a nucleic acid sequencing instrument are also disclosed.

### PRIORITY CLAIM

This application claims the benefit of priority to U.S. Provisional Application No. 61/094,785, filed Sep. 5, 2008, entitled “Instrument Validation, Calibration and Normalization Using Synthetic Beads,” which is incorporated by reference in its entirety herein.

### FIELD

The present teachings relate to nucleic acid sequence controls used for the validation, calibration, and normalization of nucleic acid sequencing instrumentation and data.

### BACKGROUND

Upon completion of the Human Genome Project, the focus of the sequencing industry has shifted to finding higher throughput and/or lower cost sequencing technologies, sometimes referred to as next generation sequencing technologies. In making sequencing higher throughput and/or less expensive, the goal is to make the technology more accessible for sequencing. These goals may be reached through the use of sequencing platforms and methods that provide sample preparation for larger quantities of samples of significant complexity, sequencing larger numbers of complex samples, and/or a high volume of information generation and analysis in a short period of time. Various methods, such as, for example, sequencing by synthesis, sequencing by hybridization, and sequencing by ligation are evolving to meet these challenges.

A disadvantage that may occur in these next generation sequencing techniques is the rise of additional system noise or performance variation for each step. At each step, system noise or performance variation for that step may be contributed from at least one of hardware, chemistry, and software. The complexity of the next generation sequencing techniques and platforms may require a variety of controls to ensure consistency of performance from sample preparation through sample sequence determination. Thus, it may be desirable to have controls or methods that separate or identify the system noise or variable (e.g., poor) performance for each step. The reduction of noise or variation may improve the normalization of data sets generated over time, providing that the vast amount of information generated can be meaningfully compared.

One conventional control uses a library of fragments created from a well-known sample, such as, for example, a strain of E. coli, and performing the sequencing method on the library of fragments. The use of naturally occurring samples for a control, however, can exhibit variation itself due to mutations within individual strands of the sample. Moreover, the preparation of these conventional controls can result in differing sequences being introduced within a desired monoclonal population of control sequences, thereby generating noise within the control system itself.

Accordingly, there is a need in the art of next generation sequencing for control systems and methods that may provide for the systematic determination and characterization of the various sources of system noise and/or degradation of performance. One desirable aspect of providing consistency in sequence determination includes providing controls that ensure instrument performance; both run-to-run, as well as instrument-to-instrument. Further, it may be desirable to provide a control technique that minimizes the potential for the detection of performance variation and/or noise during sequencing that is due to chemistry and/or library construction, and thereby permit such detection to be attributed to instrument quality.

### BRIEF DESCRIPTION OF THE DRAWINGS

FIG. 1 depicts a block diagram representing various embodiments of instrumentation used for next generation sequencing;

FIG. 2 is a schematic depiction of an exemplary embodiment of a synthetic control bead useful for validation, calibration, and normalization in nucleic acid sequencing in accordance with the present teachings;

FIG. 3 is a schematic depiction of another exemplary embodiment of a synthetic control bead in accordance with the present teachings;

FIGS. 4A-4D show a series of graphs depicting the controllable nature of a method for making various embodiments of synthetic control beads in accordance with the present teachings;

FIG. 5 is a graph showing template density results for various synthetic control beads prepared in accordance with exemplary embodiments of the present teachings;

FIG. 6 is a graph depicting the reproducibility of an exemplary embodiment of a method for making various embodiments of synthetic control beads;

FIG. 7 is an error chart generated using a synthetic control bead on an instrument used for sequencing;

FIGS. 8A-8B show two graphs demonstrating the system noise contribution of instruments used for sequencing compared to the intrinsic system noise generated using a synthetic control bead according to various exemplary embodiments of the present teachings; and

FIG. 9 is a satay plot showing the intensity of four dyes in quality control (QC) sequencing of a set of synthetic control beads comprising the same number of each of 1024 nucleic acid sequences.

It is to be understood that the figures are not drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DETAILED DESCRIPTION

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with the embodiments provided herein, the following terms, unless otherwise indicated, shall be understood to have the following meanings:

The phrase “next generation sequencing” refers to non-Sanger-based sequencing technologies having increased throughput, for example with the ability to generate hundreds of thousands of relatively small sequence reads at a time. Some examples of next generation sequencing techniques include, but are not limited to, sequencing by synthesis, sequencing by ligation, and sequencing by hybridization. Some relatively well-known next generations sequencing methods further include pyrosequencing developed by 454 Corporation, the Solexa system, and the SOLiD (Sequencing by Oligonucleotide Ligation and Detection) developed by Applied Biosystems (now Life Technologies, Inc.).

The phrase “synthetic bead” or “synthetic control bead” refers to a bead having multiple copies of a synthetic template nucleic acid sequence attached to the bead. A linker sequence may be used to attach the synthetic template to the bead.

The phrase “fragment library” refers to a collection of nucleic acid fragments generated by cutting or shearing a larger nucleic acid into smaller fragments. Fragment libraries may be generated from naturally occurring nucleic acids, such as bacterial nucleic acids. Libraries comprising similarly sized synthetic nucleic acid sequences may also be generated to create a synthetic fragment library.

The phrase “mate-pair library” refers to a collection of nucleic acid sequences generated by circularizing fragments of nucleic acids with an internal adapter construct and then removing the middle portion of the nucleic acid fragment to create a linear strand of nucleic acid comprising the internal adapter with the sequences from the ends of the nucleic acid fragment attached to either end of the internal adapter. Like fragment libraries, mate-pair libraries may be generated from naturally occurring nucleic acid sequences. Synthetic mate-pair libraries may also be generated by attaching synthetic nucleic acid sequences to either end of an internal adapter sequence.

The phrase “synthetic nucleic acid sequence” and variations thereof refers to a designed and synthesized sequence of nucleic acid. For example, a synthetic nucleic acid sequence may be designed to follow rules or guidelines. A set of synthetic nucleic acid sequences may, for example, be designed such that each synthetic nucleic acid sequence comprises a different sequence and/or the set of synthetic nucleic acid sequences comprises every possible variation of a set-length sequence. For example, a set of 64 synthetic nucleic acid sequences may comprise each possible combination of a 3 base sequence, or a set of 1024 synthetic nucleic acid sequences may comprise each possible combination of a 5 base sequence.

The phrase “control set” refers to a collection of nucleic acids each having a known sequence wherein there is a plurality of differing nucleic aced sequences. A control set may comprise, for example, beads having nucleic acid sequences attached thereto. The source of the nucleic acid sequences may be synthetically derived nucleic acid sequences or naturally occurring nucleic acid sequences. The nucleic acid sequences, either naturally occurring or synthetic, may be provided, for example, as a fragment library or a mate-pair library, or as the analogous synthetic libraries. The nucleic acid sequences may also be in other forms, such as a template comprising multiple inserts and multiple internal adapters. Other forms of nucleic acid sequences may include concatenates.

The term “template” refers to a nucleic acid sequence attached to a solid support, such as a bead. For example, a template sequence may comprise a synthetic nucleic acid sequence attached to a solid support. A template sequence also may include an unknown nucleic acid sequence from a sample of interest and/or a known nucleic acid sequence.

The phrase “template density” refers to the number of template sequences attached to each individual solid support.

The phrase “satay plot” refers to a projection of a 4-space plot onto a 2-dimensional plane. For example, a satay plot may depict the intensity of four different dyes in a 2-dimensional plane.

The present teachings relate to various exemplary embodiments of methods and systems for performing quality control in performing nucleic acid sequencing. For example, the present teachings contemplate synthetic control beads that may be used as a control for the validation, calibration, and/or normalization of instrumentation and chemistry (e.g., probe chemistry) used in sequencing. The present teachings further relate to methods and systems for validating, calibrating, and/or normalization of instrumentation used in sequencing.

Various embodiments of the present teachings relate to a system for performing quality control for nucleic acid sample sequencing. The system may include a set of solid supports, each solid support having attached thereto a plurality of nucleic acid sequences. The set may include plural groups of solid supports and each group may contain solid supports having the same nucleic acid sequences attached thereto, wherein the nucleic acid sequences of each group differ from each other, and wherein the nucleic acid sequences are synthetically derived.

Other exemplary embodiments of the present teachings relate to a method of preparing a quality control for performing nucleic acid sample sequencing that includes generating a plurality of synthetic nucleic acid sequences, wherein each synthetic nucleic acid sequence differs from another nucleic acid sequence, attaching each of the synthetic nucleic acid sequences to solid supports in plural groups of solid supports, wherein the solid supports in each group have the same synthetic nucleic acid sequence attached thereto, and combining each group of solid supports with the synthetic nucleic acid sequences attached to create a control set of solid supports for performing nucleic acid sample sequencing.

Additional embodiments of the present teachings further relate to a method of performing nucleic acid sequencing validation, the method including placing a set of solid supports each having a plurality of synthetic nucleic acid sequences attached thereto in a detection area of a nucleic acid sequencing instrument, wherein the set of solid supports comprises plural of groups of solid supports each of the solid supports in a group having the same synthetic nucleic acid sequences attached thereto and the solid supports in differing groups having differing synthetic nucleic acid sequences attached thereto. The method may further include generating a focal map to identify the location of each solid support relative to the detection area of the nucleic acid sequencing instrument, and performing one or more ligation cycles to attach a dye-labeled probe sequence to the nucleic acid sequences attached to the solid supports. The method may further include detecting the dye-labeled probes attached to each of the nucleic acid sequences, measuring the intensities of the dye-labeled probes, and comparing the measured intensities to a threshold value to determine if the instrument is functioning validly.

In the various examples and embodiments described herein, the synthetic control systems and methods are described with regard to sequencing-by-ligation systems using two-base, or dibase, encoding (e.g., as employed in SOLiD sequencing). However, as one skilled in the art would readily appreciate, the synthetic beads and methods described herein can be applied to other sequencing systems or detection techniques. The principles of synthetic control beads and methods using the synthetic beads can be applied to other systems and methods without departing from the scope of the present teachings as described herein.

Various embodiments of platforms for next generation sequencing may include components as displayed in the block diagram of FIG. 1. According to various embodiments, instrument 100 may include a fluidic delivery and control unit 110, a sample processing unit 120, an optical unit 130, and a data acquisition, analysis and control unit 140. Various embodiments of instrumentation, reagents, libraries and methods used for next generation sequencing are described in U.S. Patent Application Publication No. 2007/066931 (application Ser. No. 11/737,308) and U.S. Patent Application Publication No. 2008/003571 (application Ser. No. 11/345,979) to McKernan, et al., which applications are incorporated herein by reference. Various embodiments of instrument 100 may provide for automated sequencing that can be used to gather sequence information from a plurality of sequences in parallel, i.e., substantially simultaneously. In various embodiments of instruments and methods for sequencing, the target sequences may be arrayed or otherwise distributed on a substantially planar substrate, or plate, located in a flow cell, as will be discussed in more detail subsequently.

In FIG. 1, embodiments of an automated sequencing instrument 100 may have a sample processing unit 120 that comprises a moveable stage and a thermostatted flow cell. According to various embodiments of an automated sequencing instrument 100, a flow cell may comprise a chamber that has input and output ports through which fluid can flow. The flow of fluid may be controlled by the fluidic delivery and control unit 110, thereby allowing for the automated removal or addition of various reagents from moieties (e.g., templates, microparticles, analytes, etc.) located in the flow cell. According to various embodiments of instrument 100, a flow cell includes a location at which a substrate or plate, e.g. a substantially planar substrate or plate such as a glass slide, can be mounted so that fluid flows over the surface of the substrate or plate, and a window to allow illumination, excitation, signal acquisition, etc. using various embodiments of an optical unit 130. In various embodiments of next generation sequencing systems, moieties such as microparticles are typically arrayed or otherwise distributed on the substrate before it is placed within the flow cell.

In various embodiments of instrument 100, an optical unit 130 may comprise a source, a COD camera, and a fluorescence microscope. It will be appreciated by one skilled in the art that in various embodiments of optical unit 130, substitutions of components can be made. For example, alternative image capture devices can be used. Additionally, data acquisition, analysis and control unit 140 provides control to properly sequence various components of unit 110-140 shown in FIG. 1, such as the pumps, stage, cameras, filters, temperature control and to annotate and store the image data. A user interface is provided to assist the operator in setting up and maintaining the instrument, and may include functions to position the stage for loading/unloading slides and priming the fluid lines. Display functions may be included, for example, to show the operator various running parameters, such as temperatures, stage position, current optical filter configuration, the state of a running protocol, etc. In various embodiments, data acquisition, analysis and control unit 140 also comprises an interface to the database to record tracking data such as reagent lots and sample IDs.

It will be appreciated by one skilled in the art that various embodiments of instrument 100 can be used to practice a variety of sequencing methods including both the ligation-based methods described herein and other solid phase sequencing methods including, for example, but not limited by, sequencing by synthesis methods. As is the case for the ligation-based sequencing methods, sequencing by synthesis may be done on templates immobilized directly in or on a semi-solid support, templates immobilized on microparticles in or on a semi-solid support, templates attached directly to a substrate, etc.

According to various embodiments of the present teachings, a set of controls may include a plurality of synthetic beads each having at least one synthetic nucleic acid sequence attached thereto. In further embodiments, each synthetic bead has a plurality of the unique nucleic acid sequence attached thereto. By way of non-limiting example, the set of controls may comprise 64 groups of beads, wherein each group of beads comprises multiple copies of a respective unique nucleic acid sequence. In at least one further embodiment, the nucleic acid sequence attached to each bead consists essentially of the unique nucleic acid sequence. For example, one synthetic bead in the set may comprise the sequence 5′-AAA-3′ and another synthetic bead in the set may comprise the sequence 5′-AAT-3′, or any one of the other 63 variations possible with a 3 base sequence in the example of a 64 bead set. A set of control beads may include a multiple copies of each of a plurality of synthetic beads comprising the unique nucleic acid sequences; in other words, each set may include plural groups of beads, with each bead in a group having the same unique synthetic nucleic acid sequence attached thereto.

In at least one embodiment, the number of synthetic nucleic acid sequences may be designed based on the number of bases covered by a probe sequence used in the sequencing technique. For example, for a probe sequence that covers 3 bases at a time, a group of 64 unique synthetic nucleic acid sequences may be designed. Likewise, for a probe sequence that covers 4 bases at a time, a group of 256 unique nucleic acid sequences may be used, and for a probe sequence that covers 5 bases at a time, a group of 1024 unique nucleic acid sequences may be used. Similarly, larger groups of unique nucleic acid sequences may be used for probe sequences that cover a greater number of bases. The number of bases covered by a probe sequence may be selected, for example, based on the complexity of the analysis and the level of accuracy desired. Those having ordinary skill in the art will appreciate that probe lengths of 2 or more bases may be used and the synthetic nucleic acid sequences designed accordingly.

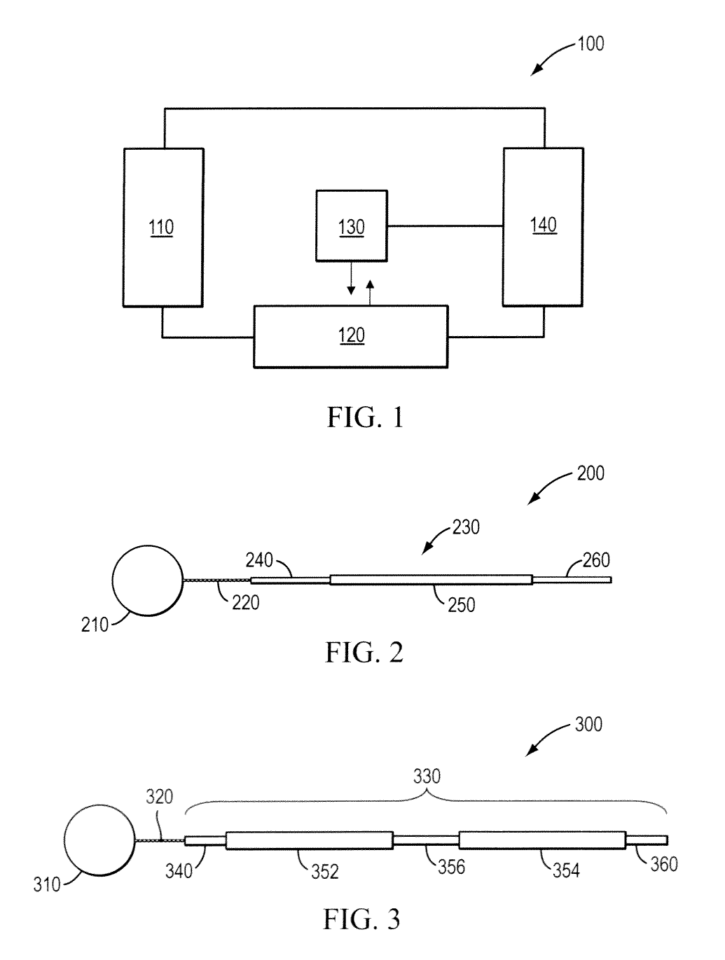

As depicted in FIG. 2, various embodiments of synthetic beads 200 include a bead 210 having a linker 220, which is a synthetic sequence for attaching a synthetic template 230 to the bead. The synthetic template 230 may include a first or P1 priming site 240, an insert 250, and a second or P2 priming site 260. The length of the linker 220 and synthetic template 230 may vary in length. For example, the length of the linker 220 may range from 10 to 100 bases, for example, from 15 to 45 bases, such as, for example, 18 bases (18 b) in length. Linker 220, which comprises P1 240, insert 250, and P2 260, may also vary in length. In at least one embodiment, P1 240 and P2 260 may each range from 10 to 100 bases, for example, from 15 to 45 bases, such as, for example, 23 bases (23 b) in length. The insert 250 may range from 2 bases (2 b) to 20,000 bases (20 kb), such as, for example, 60 bases (60 b). In at least one embodiment, the insert 250 may comprise more than 100 bases, such as, for example, 1,000 or more bases. In various embodiments the insert may be in the form of a concatenate, in which case, the insert 250 may comprise up to 100,000 bases (100 kb) or more.

In at least one embodiment, the insert 250 comprises a specifically designed synthetic sequence. Each control set comprises a plurality of synthetic beads 200 comprising different inserts 250. For example, in at least one embodiment, a control set may comprise synthetic beads comprising at least 1024 unique inserts 250. According to various alternative embodiments, a control set may comprise 64 unique inserts, 256 unique inserts, or more. For example, for an insert comprising a unique sequence of 5 bases (5 b), also known as a pentamer, chosen from the four standard bases (A, G, C, and T), a total of 4⁵ or 1024 unique sequences may be used. One of ordinary skill in the art would recognize that the number of bases in each unique insert sequence 250 may be selected based on several criteria, including, but not limited to, the desired accuracy of the control set, the complexity of the sample being studied, etc.

In at least one embodiment, a control set of synthetic beads may include beads that have additional unique nucleic acid sequences attached thereto. By way of example, additional unique synthetic acid sequences may be introduced to account for any biases that are noticed after the generation of a set of controls so as to augment the controls and form a control set that accounts for that bias. For example, when dibase sequencing is used to analyze the control set with probes that cover 5 bases at a time, a set of 1024 unique nucleic acid sequences would provide every possible pentamer combination of the 4 standard bases at a given location on the insert. Although the 1024 unique nucleic acid sequences can provide every possible pentamer combination at a given location on the insert, additional beads associated with additional unique nucleic acid sequences may also be provided. While not wishing to be limited by theory, it is believed that biases may exist in certain sequences or at certain locations within each synthetic nucleic acid sequence, such as at junctions between pentamer sequences in the above example, where a junction is defined as the last base of a first pentamer and the first base of a second pentamer interrogated by a probe covering 5 bases at a time. In at least one embodiment, additional beads comprising synthetic nucleic acid sequences similar to any nucleic acid sequence that exhibits a bias during testing may also be included.

In various exemplary embodiments, the number of possible additional synthetic nucleic acid sequences may be up to the number of unique nucleic acid sequences in the control set squared to account for each ligation event spanning the junction between two adjacent probed sequences (e.g., two adjacent pentamers for the 5-base probe sequences example described above). For example, a control set comprising 64 unique synthetic nucleic acid sequences may comprise a total of 64²=4,096 different probe sequences to cover the entire set of interactions between ligation events. Similarly, a control set comprising 1024 unique probe sequences may comprise a total of 1024²=1,048,576 sequences to cover the entire set of interactions between ligation events.

According to various exemplary embodiments of the present teachings, the control set may comprise a plurality of beads 200 each comprising a unique insert 250 chosen from 1024 unique inserts comprising a unique pentamer at every 5 bases of the ligation cycle when interrogating 5 bases at a time with a probe sequence in 2-base encoding. Each of the plurality of beads 200 may comprise a plurality of copies of each insert 250, such as, for example, an average of 5,000 copies to 250,000 copies of the insert 250, for example, an average of 95,000 copies to 170,000 copies. In at least one embodiment, the beads 200 may have an average of about 130,000 copies of the insert 250. One skilled in the art would recognize that the number of copies of the insert 250 may vary depending on the experiment being run, and the actual number may be more or less to meet the needs of various applications.

In dibase sequencing, a probe sequence interrogates a set number of bases during each of a plurality of ligation cycles. For example, a probe sequence that covers 5 bases at a time will cover the first 5 bases, followed by the second set of 5 bases, etc., during each subsequent ligation cycle. When dibase sequencing is used with a 5 base probe sequence, only 2 of the 5 bases covered by the probe sequence are interrogated by the probe. In various embodiments, other probes may be used that interrogate more bases (e.g., multibase sequencing) or have different ratios of bases that interrogate the synthetic nucleic acid sequence compared to bases that do not interrogate, such as, for example, a dibase probe covering 4 bases and interrogating 2 bases of the synthetic nucleic acid sequence. To build a complete data set, at least the same number of primers as the number of bases covered by each probe sequence should be used, wherein each primer is off-set by one base. For example, a 60 base insert would require 12 ligation cycles using 5 primers off-set from one another to provide data sufficient to identify each base when using a probe that interrogates 5 bases at a time. Thus, when using a probe that interrogates x bases at each ligation cycle, the number of ligation cycles required is equal to the length in bases, l, divided by x and rounded up to the next whole number. In at least one embodiment, each unique pentamer associated with each insert appears only once in that insert. The sequence interrogated by subsequent probes on a single template should not repeat. In other words, the template sequence may be designed such that a sequence of any x bases in a row are not repeated in any other series of x bases in a row at a distance of n multiplied by x away, wherein n is a positive integer. For example, a unique pentamer (i.e., x=5) appearing in the first 5 bases of the insert will not appear in each of the consecutive, subsequent 5-base sequences in the remainder of the insert, i.e., the 5-base sequences a multiple of x away, such as 5 bases, 10 bases, 15 bases, etc.

According to at least one embodiment, the remainder of the insert sequence excluding the pentamer may avoid quasi-repetitive sequences that are similar to the pentamer. For example, if the first pentamer for a bead is the sequence AAAAA, the remainder of the insert sequence may avoid similar sequences, such as, for example, AAAATAAAACAAAAG. When the synthetic beads are used with dibase encoding (also referred to as 2-base encoding), with which those ordinarily skilled in the art are familiar, the synthetic sequence insert may also be designed to avoid repeating the same color call between neighboring ligation cycles to possibly aid in the distinction of residue signals from the previous ligation cycles. Thus, for example, when using fluorescent dye tags to encode for bases (either individually or as combinations), the synthetic sequence insert may be designed so that if one color is detected during a first sequencing cycle (e.g., ligation cycle), the next sequencing cycle will not yield the same color. In various exemplary embodiments, therefore, if during consecutive probe sequencing cycles the same color is detected, it may be determined that an error has occurred in the sequencing process and the entire sequencing run may be aborted if necessary.

According to various embodiments of the present teachings, the synthetic template sequences may be chosen as those sequences that have a minimum folding free energy from randomly generated sequences. For example, a large number of sets of sequences, such as, for example, 10,000 generated sets of 1024 synthetic sequences, may be analyzed to determine the sequences having the lowest free energy, for example using software and/or other techniques useful for calculating folding free energy. Potential secondary structure issues may also be avoided when selecting the synthetic template sequences. Some sequences in the set may be randomly selected to manually check for potential secondary structure issues. In at least one embodiment, the random template sequences may be determined using the following algorithm. For a control set that comprises 1024 different sequences, all 1024 pentamers are generated in random order as the seed of the sequences. Next, each of the 1024 sequences are extended by the following rules: 1) group all 1024 sequences by the last 4 bases, which should result in 256 groups and 4 sequences in each group; 2) extend different bases A, T, G, and C randomly to the 4 sequences, resulting in all four sequences being appended to with different bases; 3) check if the extended sequences satisfy any required constraints; if the required restraints are satisfied, repeat step 2 for another group, and if the required restraints are not satisfied, then step 2 can be repeated for a prescribed number of retries (e.g., up to 4! or 24 combinations that need to be tested); 4) if the constraints cannot be satisfied after reaching the prescribed number of retries, start with a new set of 1024 randomly generated pentamer sequences; 5) if the constraints are satisfied for all groups, repeat the process from step 1 for all groups to extend another base; and 6) once the desired length has been reached, output the resulting synthetic sequences.

An alternative exemplary embodiment of a synthetic bead 300 is schematically shown in FIG. 3. The synthetic bead 300 may comprise a bead 310, a linker 320, and a synthetic template 330. The synthetic template 330 of synthetic bead 300 may be analogous to a mate pair library construction. Synthetic template 330, may comprise a first or P1 priming site 340 and second or P2 priming site 360, which may range in length from 10 to 100 bases, for example, from 15 to 45 bases, such as, for example, 23 b in length. Synthetic template 330 further comprises an insert 350, which may comprise a first synthetic tag sequence 352, a second synthetic tag sequence 354, and an internal adapter 356 located between the first and second tag sequences 352, 354. The first and second tag sequences 352, 354 may have a length ranging from 2 bases (2 b) to 20,000 bases (20 kb), such as, for example, 60 bases. The first and second tag sequences 352, 354 may be the same sequence or different sequences. The first and second tag sequences 352, 354 may comprise a different number of bases or the same number of bases. The internal adapter 356, which may be common to all template sequences in a control set, may have a length ranging from 10 to 100 bases, for example, from 15 to 45 bases, such as, for example, 36 bases.

The first and second template sequences 352, 354 may comprise a specifically designed synthetic sequence. In at least one embodiment, a control set may comprise a plurality of synthetic beads 300, each of which comprises a unique sequence, such as described above, in the first and second tag sequences 352, 354. In at least one embodiment, each of the synthetic beads 300 comprises a unique sequence chosen from 1024 unique sequences (4⁵ possible pentamer sequences). The sequences of the first and second tag sequences 352, 354 may be selected based on the design rules described above. Additionally, the bases in the first tag sequence 352 and the bases of second tag sequence 354 may be chosen to avoid quasi-repetitive sequences similar to pentamer sequence.

In various embodiments, additional internal adapters and tag sequences may be used. For example, an insert may comprise 3 or more tag sequences and 2 or more internal adapters, respectively, in an alternating pattern. Various other types of sequence patterns may be utilized for the synthetic nucleic acid sequences depending on the desired application.

In at least one embodiment, the internal adapter may comprise a primer sequence, which may be an additional primer in a PCR amplification process.

In at least one embodiment, the synthetic beads having various synthetic template designs may be prepared and attached to a solid support using PCR (polymerase chain reaction). Any known method of PCR may be used to amplify and attach the nucleic acid sequences to the solid supports. In at least one embodiment, each synthetic template design can be amplified in a separate PCR solution. In this manner, each unique synthetic template sequence, such as a template sequence comprising a unique pentamer, may be amplified in a linear growth fashion onto beads in individual batches. As a result, all bead batches prepared in separate PCR solutions may be monoclonal, which may reduce polyclonal and non-specific amplification sample preparation noise that may otherwise be present in controls prepared using other methods.

In one exemplary embodiment, to prepare a set of synthetic beads having 1024 unique template sequences, 11 or more 96-well plates can be used to support 1024 separate reactions in each well. For example, 1 unique synthetically derived template (e.g., a synthetic sequence obtained using the methodology described above) may be placed in each of 1024 wells and on the order of a hundred thousand or more beads may be placed in each well. The number of beads in each well may vary depending on the amount of beads needed to achieve sufficient templating of the beads (i.e., the number of beads having a sufficient template density attached thereto). For example, the number of beads may range from 200 million to 1 billion or more. As one skilled in the art would readily appreciate, the actual number of beads that are used and that may be templated in each PCR batch could be more or less and may depend, for example, on the size of the reaction vessel in which each PCR reaction takes place; those having ordinary skill in the art would understand that individual PCR reaction volumes can range from nanoliters to liters. PCR on the well-plates may be performed for a number of cycles selected so as to achieve a desired template loading of the synthetic sequences on the beads in the wells. In various exemplary embodiments, as discussed above, the PCR cycles may be repeated to achieve an average template loading ranging from about 5,000 copies to about 250,000 copies per bead, for example, from about 95,000 to about 170,000 copies per bead.

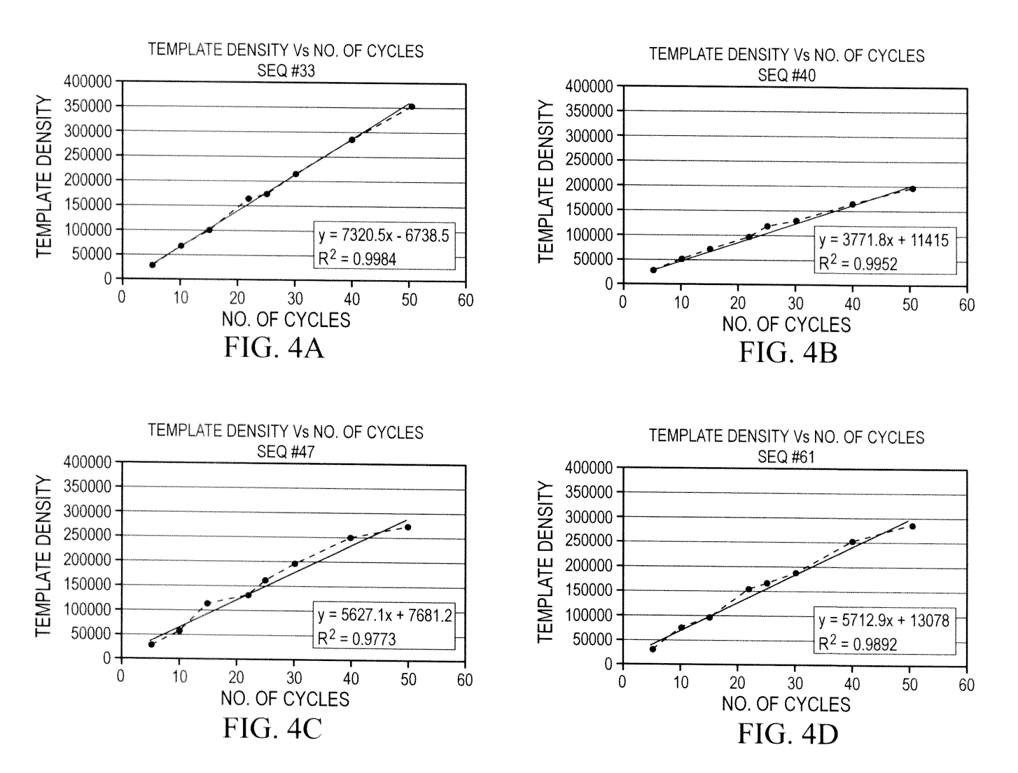

For example, according to various embodiments, synthetic beads may be prepared using beads having a P1 priming site, and amplifying each of a number of specifically designed templates in individual batches using PCR. The process is a linear amplification, and not an exponential amplification, as depicted in FIGS. 4A-4D. In FIGS. 4A-4D, the template density as a function of the number of thermal cycles is shown for a cross-section of templates of different sequences. Though the rate of incorporation of template at available P1 sites may vary for the different template designs, the rate of incorporation in all cases may proceed in a linear fashion, thus allowing control over the template density for each batch.

Although PCR can be used to amplify and attach the synthetic nucleic acid sequences to the solid supports, such technique should be understood as non-limiting and exemplary. In various alternative embodiments of the present teachings, the nucleic acid sequences may be attached to a solid support either chemically or biochemically. For example, the nucleic acid sequences may be attached by chemically forming a covalent bond to the solid support or to a linker attached to the solid support. In another example, the nucleic acid sequence may be attached to the solid support or a linker attached to the solid support enzymatically.

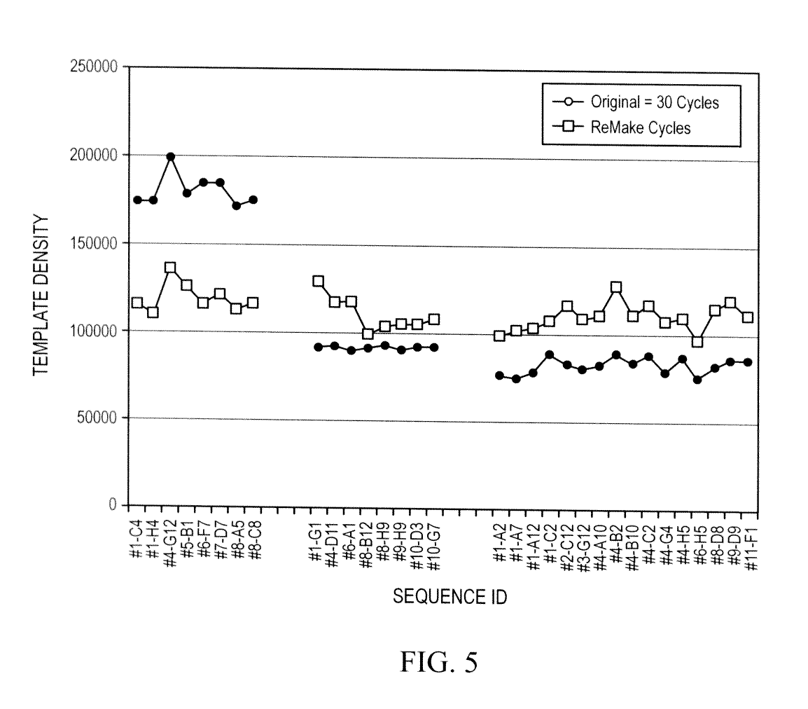

That the process for creating controls in accordance with the present teachings may be tunable is demonstrated in the graph presented in FIG. 5. In FIG. 5, a plot of template density as a function of selected templates is shown. As can be seen in the plot for the original 30 cycles, indicated for the plots using diamonds, some templates may form at a higher rate than others, which is consistent with the data shown in FIGS. 4A-4D. In the graphs indicated using squares, these are subsequent, or remake reactions. As can be seen in this plot, this demonstrates that the template density for all sequences can be normalized. Since the synthesis is linear, and may be well characterized for all synthetic template designs, the template density may be readily adjustable.

According to at least one embodiment, the individual batches may be analyzed, for example, for the number of beads in a batch, the template density (e.g., average template density), and reaction variance. Using linear amplification, the template density per bead in a batch may be monitored and precisely controlled. According to various embodiments, synthetic bead batches may be prepared with a finely tuned template density. As described above, in various exemplary embodiments of synthetic beads, an average template loading ranging from about 5,000 templates per bead to about 250,000 templates per bead may be desired. However, the tunable nature of the preparation allows for the equivalent of between about one P1 site per bead to all available P1 sites per bead. After preparation and characterization of the batches of monoclonal beads, the beads can be pooled, for example by pouring substantially equal concentrations of each of the groups of beads (e.g., 1024 groups for 1024 unique synthetic template sequences) to create a synthetic bead control set containing the substantially same number of beads comprising each unique template. Therefore, each control set may comprise roughly the same number of templates. For example, in various exemplary embodiments, a control set may comprise from 100 billion to 1500 billion beads. In at least one embodiment, a control set may comprise 800 billion synthetic beads. One skilled in the art would recognize that the number of beads in a control set may be chosen based on the application for which the control set is used and the intensity of response desired that would be provided by a greater or lesser number of beads.

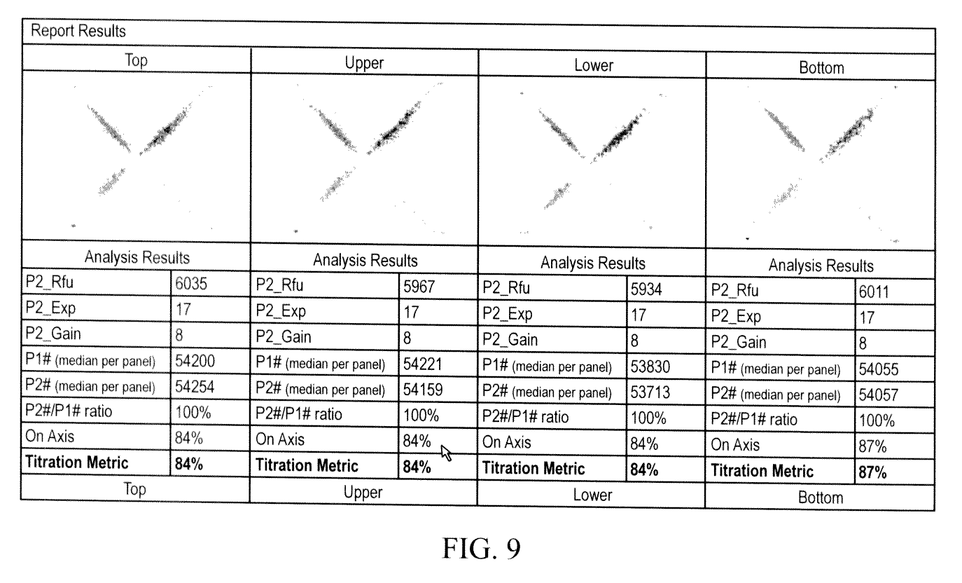

In at least one embodiment, the quality of the synthetic beads can be determined using a quality control (QC) sequencing method to verify adequate template loading and number of loaded beads. In an exemplary embodiment of a QC sequencing method, a pooled set of synthetic beads are placed on a slide (e.g., in a flow cell). A focal map is generated of the labeled P1 and P2 primers to identify the location of all of the beads, followed by a reset (e.g., removal of the P1 and P2 labels) followed by a single ligation cycle with Primer 1. No dephosphorylation or cleavage steps are carried out. The slide is then scanned. Because the beads are monoclonal and no cleavage steps are carried out, the only noise present results from an inefficient reset following the focal map or a misincorporation in the ligation step. A satay plot, such as the satay plots shown in FIG. 9, shows the intensity of each of four dyes as used in a 2-base encoding system. The four separate satay plots shown in FIG. 9 correspond to 4 different areas, or quads, of the slide. A comparison of the 4 satay plots for a slide may show the distribution of the beads on the slide. The on axis percentage shows the variation of the intensity of the dyes.

After analysis and characterization of the synthetic beads, a synthetic bead control set may be created by pooling aliquots from the individual batch preparations (e.g., for the example above, from the 1024 batches). In addition to providing that all probes may be interrogated in every round of sequencing (e.g., 1024 pentamer probes in the example provided above), other design features of various embodiments of synthetic beads include, but are not limited by, the synthetic templates have minimum secondary structure, may be designed after a fragment library, a mate pair library, or more complex library, and can be readily decoded from color to base assignment. Additionally, various embodiments of synthetic beads may be prepared using various methods that provide scaling of production using a controllable process, as well as providing that the template density is tunable. These methods of preparation ensure that various embodiments of synthetic beads are highly reproducible from batch-to-batch and may be finely tuned based on differences in template length or complexity for normalization of batches.

Various embodiments of the synthetic beads may be used in a variety of solid phase sequencing systems, as previously described. In that regard, various embodiments of synthetic beads may be used in any of the previously mentioned next generation sequencing methods such as, but not limited by, sequencing by synthesis, sequencing by hybridization, and sequencing by ligation.

For example, one approach to sequencing by ligation uses 2-base encoding, as described by McKernan, et al. in the previously mentioned incorporated references. According to various embodiments of sequencing by ligation using 2-base encoding, probes of 8 b in length may be used, in which the first three bases are degenerate, and the last three are universal. The fourth and fifth bases are the two bases being interrogated. In various embodiments of 2-base encoding methods, four different dye tags may be used for detecting the probes. Therefore, a single color limits the potential dinucleotide to being four out of sixteen possible combinations. During the ligation process, the three universal bases bearing the fluorescent tag are cleaved, yielding a detectable fluorescent signal, so that in each cycle, a pentamer of bases is added to the growing chain. For various embodiments of methods for sequencing by ligation utilizing such an approach, there would be 1024 possible pentamer probes. In various embodiments of the present teachings, probes of other lengths may also be used. For example, probes having a length of 2 or more bases may be used in at least one embodiment.

Using the above example of pentamer probes, various embodiments of monoclonal synthetic beads may be designed to interrogate all 1024 possible pentamer probes in every round of sequencing. For example, 1024 specific monoclonal template designs may be separately prepared, for example, using individual PCR reactions. Such monoclonal bead and probe combinations may also be used for multibase encoded sequencing where greater than 2-base encoding is utilized, and those having ordinary skill in the art would understand how to modify the design of the synthetic sequences to be useful with multi or single-base encoding sequencing techniques Further, when using dibase encoding wherein four fluorescent dye tags (e.g., four colors) are used to encode for the sixteen possible two base combinations and thus each color represents four potential two based combinations, the synthetic sequence inserts of synthetic control beads may be designed so that if one color is detected during a first ligation cycle, the next ligation cycle will not yield the same color.

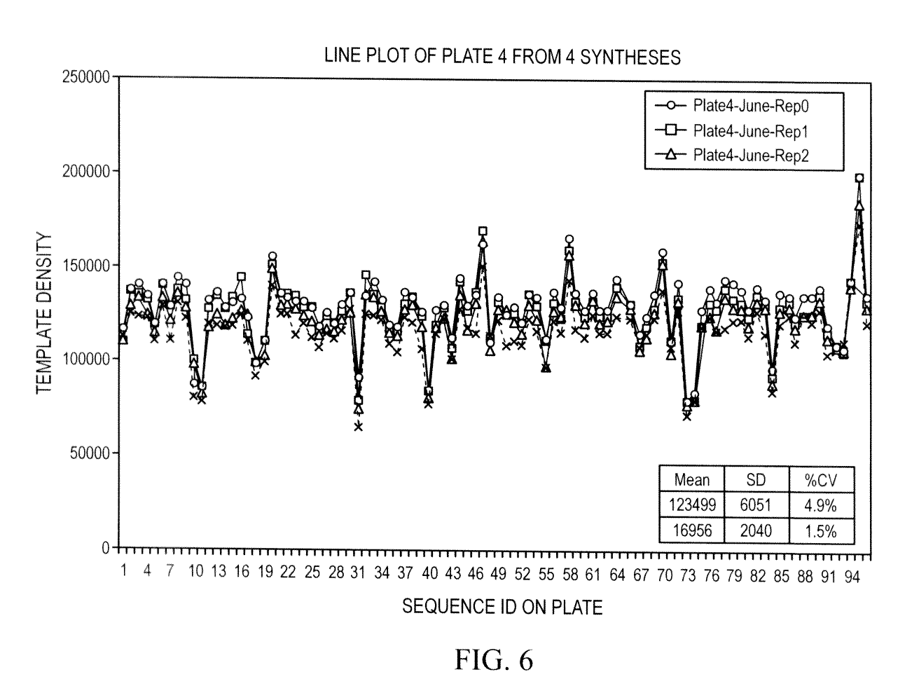

That various embodiments of synthetic beads have the attributes as a control for evaluating instrument function is demonstrated in FIGS. 6-8. The data used as the basis for these graphs was generated using a sequence by ligation method as previously described, on an instrument as depicted and described for FIG. 1.

The overall reproducibility of various embodiments of synthetic bead batches is demonstrated in FIG. 6, which is a graph of template density versus sequence ID. A single plate placed in the flow cell was subdivided to accommodate synthetic bead controls from four batches, so that the sequencing was run simultaneously for the four batches. The batches produce data that are substantially superimposed, with a coefficient of variation, expressed as percent (CV %) under 5%.

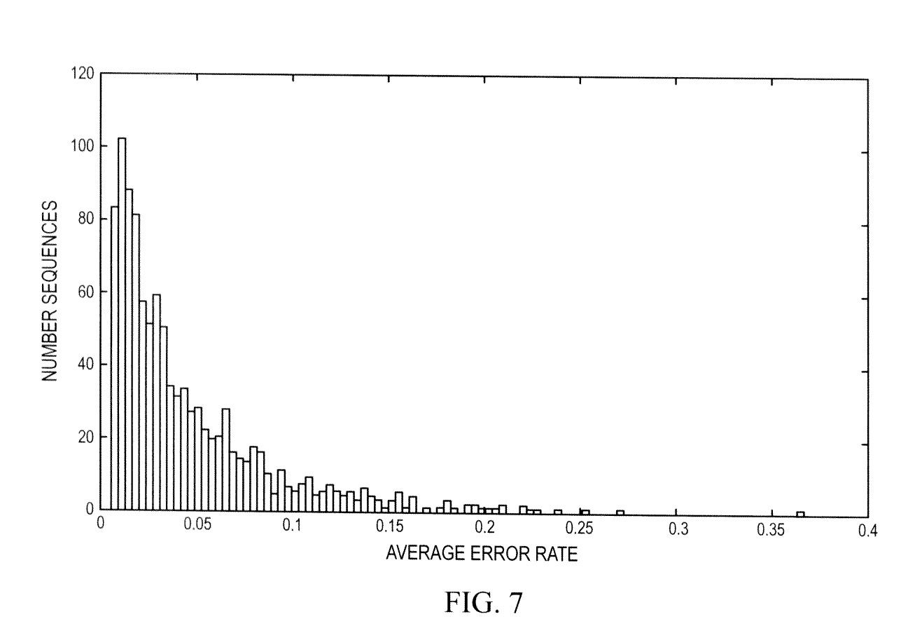

The error rate determination for sequencing may be an important metric for characterizing instrument performance, but only under the conditions that the errors in sequencing are primarily a function of instrument performance, and not the sample being sequenced. Unlike other controls that have polyclonal features (e.g., polyclonal sequences attached to beads), various embodiments of synthetic beads in accordance with the present teachings can be used to determine an error rate plot as shown in FIG. 7. According to various embodiments of synthetic beads, the sequences for the synthetic templates can be readily assigned in contrast to the assignment of sequences for polyclonal beads. Therefore, various embodiments of synthetic beads may have a reproducible error rate, as shown in FIG. 7.

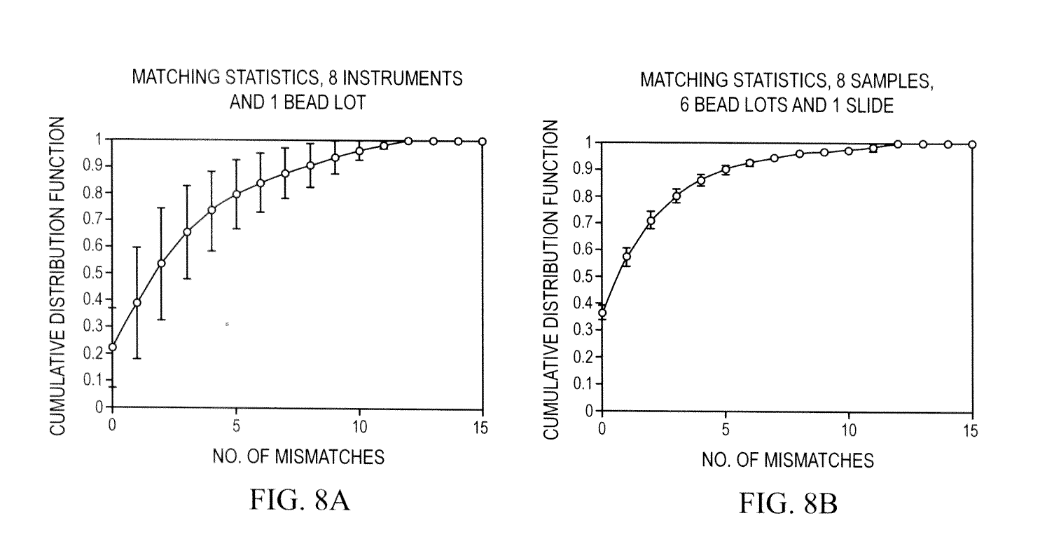

FIGS. 8A and 8B demonstrate that the error rate in sequencing when using various embodiments of synthetic bead controls may be attributed to the instrument function and not the bead chemistry. In the data presented in Graph I, the statistically determined error bars are shown for data collected from 8 instruments in the plot of cumulative distribution function versus number of mismatches. In the data presented in Graph II, the comparative data is shown for 8 bead samples drawn from 6 bead lots on a single slide for one instrument. The contribution to system noise by the beads is 10% that contributed by the instrumentation. Based on these data, there is only about a 1% chance that an instrument could fail quality control evaluation as a result of bead variability. In that regard, various embodiments of synthetic beads, which contribute such a small portion of the overall system noise, may be used in methods for instrument quality and validation, where a metric, such as the system noise or error rates generated using the beads can be compared to a predetermined limit of acceptable performance for that metric.

In at least one embodiment, the control set of synthetic beads can be used to determine the quality and efficacy of the dye-labeled probe sequences. The dye response exhibits a linear response to the concentration of the dye-labeled probe sequences. Therefore, the quality of a batch of dye-labeled probe sequences may be tested using the synthetic beads described above. Likewise, comparisons between different dye-labeled probe sets can be made. In at least one embodiment, the quality of unlabeled probes can be monitored with subsequent ligation cycles with dye-labeled probes or by mixing a known ratio of labeled and unlabeled probes.

According to various embodiments of synthetic beads, the design features of the synthetic beads, as well as the methods of preparation of synthetic beads ensuring batch-to-batch reproducibility make synthetic beads ideal controls for instrument validation, calibration, and normalization, as well as for probe chemistry quality control.

According to various embodiments of the present teachings, control sets of the synthetic beads described above may be used to validate sequencing instruments, for example, for verifying instrument quality (IQ). In at least one embodiment, QC sequencing runs, as described above, may be performed before and after an experimental sequencing run. The results from the QC sequencing run before the experimental run and the results from the QC sequencing run after the experimental run may be compared to determine whether the instrument functioned properly. For example, if the QC sequencing run performed after the experimental run differs from the QC sequencing run performed before the experimental sequencing run, the results of the experimental run may be suspect due to changes in the instrument's performance.

According to at least one embodiment, the synthetic beads may be used to determine the distribution of beads (both control and thus beads with target sequences), for example, on a slide or flow cell. For example, an ideal group of satay plots measuring different areas of a slide should depict substantially evenly distributed scatter along each axis. If an instrument is malfunctioning, a comparison of satay plots for each area may identify an error with the instrument. Additionally, a control set of the synthetic beads may be used to show that the beads in an experimental sequencing run were evenly distributed.

In at least one other embodiment, a set of synthetic control beads may be used to determine overall (i.e., aggregate) matching statistics. The overall matching statistics may be used to assess the quality of each sequencing run. For example, a low mismatching rate may indicate that the quality of the sequencing run was satisfactory, while a high mismatching rate may indicate poor run quality. Individual matching rates of each of the unique template nucleic acid sequences also may be used to detect sequence context dependent issues, such as, for example, poor probe chemistry and/or systematic ligation and/or hybridization issues. In using synthetic sequences, the ambiguity of mapping the sequence reads to the reference (control) is removed, permitting the measurement of the performance on each of the individual sequences to be more consistently determined.

In at least one embodiment, the IQ sequencing runs may first be tested on a set of test SOLiD sequencers (generally 30-40) (e.g., SOLID sequencers commercially available from Life Technologies, Inc.) containing both passed and faded instruments. These instruments may be predetermined as pass or fail in advance of the IQ sequencing runs. The specifications of a passing instrument may be set as the mean matching percentage minus one and a half standard deviation, which mathematically covers 95% of the passing instruments. For example, the matching percentage specification of a passing instrument in accordance with an exemplary embodiment may be set at 77.7%; in other words, the matching percentage may be greater than about 77.7% for an instrument to be deemed as having passed IQ. Similarly, the matching percentage of the individual synthetic sequences can be used to determine the quality of the control set of beads. A subset of erroneously synthesized nucleic acid templates or missing templates could be detected and observed as a block of sequences with high error rates.

In at least one embodiment, the IQ may be analyzed by comparing the intensity measured after each ligation cycle in separate runs of the control set. While not wishing to be limited by theory, it is believed that the response of the probe intensity may vary in any given sequence based on the physical position of the interrogated bases in the sequence. For example, a probe that detects a 2-base sequence near the beginning of a nucleic acid sequence may provide a different response intensity than a similar probe at the same 2-base sequence that is physically farther down the nucleic acid sequence and probed in a later ligation cycle. This variation may be reproducible and predictable. According to various embodiments, this variation may be used as an indicator of IQ. For example, if the variation in one sequencing run differs from the variation in another sequencing run of the same control set, an instrument error may be the cause of the variation and may signal a problem with an experimental sequencing run.

In at least one embodiment, the synthetic control beads also may be used to normalize data between two different instruments. Because the results of the control sets are reproducible, as shown in FIG. 7 and FIG. 8, a set of control beads could be used to determine any differences in the sensitivities of different instruments by running the QC sequencing on the different instruments. The data obtained from the QC sequencing runs can be used to normalize the data provided by each instrument.

According to at least one embodiment, the error rates for sequential ligation cycles may be used to determine errors that may have occurred in previous ligation cycles. For example, in a control set comprising 1024 unique nucleic acid sequences, the error rate of a particular color measurement may depend on the pentamer sequence of the current ligation cycle and the previous ligation cycle. Because a pentamer by itself may ligate well in one cycle, it may be erroneous when it is accompanied by certain upstream pentamers. In at least one embodiment, an interaction matrix that compares the measurements of current ligation cycles and previous ligation cycles may be used to determine errors. While not wishing to be limited by theory, it is believed that an interaction matrix of sequencing error rates may be estimated using biological sequence constructs (e.g., fragment libraries or mate-pair libraries), a synthetic sequence may provide an unbiased estimate of the interaction matrix of sequencing error rates.

Although the various embodiments described beads as the solid support on which the synthetic nucleic acid sequences are attached, other solid supports may also be utilized, such as, for example, microparticles, micro-arrays, slides, etc. Additionally, the beads may comprise any known material known for such use, including polymeric and inorganic materials, as well as paramagnetic and non-paramagnetic materials. The selection of the appropriate solid support would be within the capabilities of one of ordinary skill in the art to determine based on the sequencing platform used, the materials used to carry out the study, and any other factor that may influence the running of the experiment.

While the principles of the present teachings have been described in connection with specific embodiments of synthetic beads and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

What is claimed is:

1. A system for performing quality control for nucleic acid sample sequencing, the system comprising:
a set of solid supports, each solid support having attached thereto a plurality of nucleic acid sequences,
wherein the set comprises plural groups of solid supports and each group contains solid supports having the same nucleic acid sequences attached thereto,
wherein the nucleic acid sequences of each group differ from each other, and
wherein the nucleic acid sequences are synthetically derived.

2. The system of claim 1, wherein the solid supports are beads.

3. The system of claim 1, wherein the plurality of nucleic acid sequences are attached to each solid support via polymerase chain reaction of a template nucleic acid sequence.

4. The system of claim 1, wherein the plurality of nucleic acid sequences are attached to each solid support chemically or biochemically.

5. The system of claim 1, wherein each nucleic acid sequence is designed such that consecutive cycles of ligation during a sequencing-by-ligation process do not yield the same detected color with dye-labeled probe nucleic acid sequences.

6. The system of claim 1, wherein the set comprises at least 64 groups of solid supports.

7. The system of claim 6, wherein the set comprises at least 1024 groups of solid supports.

8. The system of claim 1, wherein each solid support has from about 5,000 to about 250,000 monoclonal nucleic acid sequences bound thereto.

9. The system of claim 1, wherein each nucleic acid sequence comprises a plurality of tag sequences, wherein the plurality of tag sequences comprise the same sequences or different sequences.

10. The system of claim 9, wherein an internal adapter sequence is disposed between each of the plurality of tag sequences.

11. The system of claim 1, wherein the nucleic acid sequences attached to the solid supports are monoclonal nucleic acid sequences.

12. The system of claim 1, wherein the nucleic acid sequences are designed such that the folding free energy of each sequence is minimized.

13. The system of claim 1, wherein the nucleic acid sequences are designed such that a sequence of any x bases in a row are not repeated in the nucleic acid sequence in another series of x bases in a row at a distance of nx away, wherein n is a positive integer and x is the number of bases covered by probe sequences during each ligation cycle in a sequencing-by-ligation process.

14. A method of preparing a quality control for performing nucleic acid sample sequencing, comprising:
generating a plurality of synthetic nucleic acid sequences, wherein each synthetic nucleic acid sequence differs from another nucleic acid sequence;
attaching each of the synthetic nucleic acid sequences to solid supports in plural groups of solid supports, wherein the solid supports in each group have the same synthetic nucleic acid sequence attached thereto; and
combining each group of solid supports with the synthetic nucleic acid sequences attached to create a control set of solid supports for performing nucleic acid sample sequencing.

15. The method of claim 14, wherein generating the plurality of synthetic nucleic acid sequences comprises generating sequences such that any x bases in a row of the sequences are not repeated in the nucleic acid sequence in another series of x bases in a row at a distance of nx away, wherein n is a positive integer and x is the number of bases covered by probe sequences during each ligation cycle in a sequencing-by-ligation process.

16. The method of claim 14, further comprising amplifying the synthetic nucleic acid sequence on each solid support so that each solid support has a plurality of monoclonal copies of the synthetic nucleic acid sequence attached thereto.

17. The method of claim 16, wherein the amplifying comprises amplifying each synthetic nucleic acid sequence in a separate reaction from the other synthetic nucleic acid sequences.

18. The method of claim 14, wherein each solid support has from about 5,000 to about 250,000 synthetic nucleic acid sequences attached thereto.

19. The method of claim 14, wherein attaching each of the synthetic nucleic acid sequences to solid supports comprising attaching the synthetic nucleic acid sequences to the solid supports chemically or biochemically.

20. The method of claim 14, wherein the solid supports are beads.

21. The method of claim 14, wherein each synthetic nucleic acid sequence is designed such that consecutive cycles of ligation during a sequencing-by-ligation process do not yield the same detected color with dye-labeled probe nucleic acid sequences.

22. The method of claim 14, wherein the combined group of solid supports comprises at least 64 groups of solid supports.

23. The method of claim 22, wherein the combined group of solid supports comprises at least 1024 groups of solid supports.

24. The method of claim 14, wherein each nucleic acid sequence comprises a plurality of tag sequences, wherein the plurality of tag sequences comprise the same sequences or different sequences.

25. The method of claim 24, wherein an internal adapter sequence is disposed between each of the plurality of tag sequences.

26. A method of performing nucleic acid sequencing validation, comprising:
placing a set of solid supports each having a plurality of synthetic nucleic acid sequences attached thereto in a detection area of a nucleic acid sequencing instrument, wherein the set of solid supports comprises plural of groups of solid supports each of the solid supports in a group having the same synthetic nucleic acid sequences attached thereto and the solid supports in differing groups having differing synthetic nucleic acid sequences attached thereto;
generating a focal map to identify the location of each solid support relative to the detection area of the nucleic acid sequencing instrument;
performing one or more ligation cycles to attach a dye-labeled probe sequence to the nucleic acid sequences attached to the solid supports;
detecting the dye-labeled probes attached to each of the nucleic acid sequence;
measuring the intensities of the dye-labeled probes; and
comparing the measured intensities to a threshold value to determine if the instrument is functioning validly.


SEQUENCE LISTING (1 sequence)

SEQ ID NO: 1 - DNA, 15 bp, Artificial Sequence
  Control sequence for validation, calibration and/or normalization
aaaataaaacaaaag